(19) United States
(12) Patent Application Publication
(10) Pub. No.: US 2006/0200027 A1
(43) Pub. Date: Sep. 7, 2006

(54) Articulated placement guide for sensor-based noninvasive blood pressure monitor

(75) Inventor: Kevin Ray Evans, New Richmond, WI (US)

Correspondence Address: ALTERA LAW GROUP, LLC, 6500 CITY WEST PARKWAY, SUITE 100, MINNEAPOLIS, MN, 55344-7704, US

(73) Assignee: Medwave, Inc.

(21) Appl. No.: 11/072,199

(22) Filed: Mar. 4, 2005

(51) Int. Cl. A61B 5/02

(52) U.S. Cl. 600/485

(57) ABSTRACT

Blood pressure may be determined from a sensor-based monitoring device that non-invasively senses pressure pulses from an underlying artery. A user positions the sensor on the surface of an anatomical structure over the-artery, such as over the edge of the radius bone of the wrist, using an articulated placement guide. The placement guide has a predetermined shape that is generally conformable with the cross-section of the anatomical structure. As hold-down pressure is applied, pressure pulses are sensed by the sensor to produce data from which the blood pressure may be obtained. The articulated placement guide has a plurality of segments which are connected by articulation regions, and is capable of expanding significantly to fit a wide range of sizes of the anatomical structure on which the monitoring is being done. In this manner, a single articulated placement guide can accommodate a variety of different patients, from children to bariatric.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention is directed to positioning of devices on anatomical structures for monitoring blood pressure, and more particularly to placement guides for sensor-based noninvasive blood pressure monitors.

2. Description of the Related Art

A fast, accurate and effective technique for measuring blood pressure, including systolic pressure, diastolic pressure, mean pressure, and pulse, is a sensor-based monitoring device that non-invasively detects pressure pulses in the radial artery. Using automated or manually applied pressure, the monitoring device presses the sensor down upon the wrist over the radial artery, so that the artery is pressed against the radius bone. The sensor should be positioned at the distal edge of the radius bone. Devices of this type and their associated methods of calculating blood pressure are described in various patents, including U.S. Pat. No. 5,797,850 issued Aug. 25, 1998 to Archibald et al., U.S. Pat. No. 5,640,964 issued Jun. 24, 1997 to Archibald et al., and U.S. Pat. No. 6,558,335 issued May 6, 2003, to Thede. Commercially available devices of the sensor-based type include the Vasotrac® noninvasive blood pressure monitor and the Vasotrax™ noninvasive blood pressure monitor, which are available from Medwave Inc. of Danvers, Mass.

FIG. 1 shows a wrist assembly 10 for a blood pressure monitoring system as described in the Archibald et al. '850 patent. The wrist assembly 10 is mounted on a patient's wrist 11 for applying a varying hold down pressure to an artery within the wrist 11, and for sensing blood pressure waveforms produced in the artery. The exemplary wrist assembly 10 includes a swivel mount 12, a hold down assembly 13, a sensor interface assembly 14, a waveform pressure transducer 15, a hold down pressure transducer 16, a connection tube 17, a wrist mount 18 and a wrist pad 19. The wrist assembly 10 is for an automatic monitor, in which the contact force applied to the sensor on the artery is controlled automatically. The contact force or “hold-down” pressure is applied by an fluid system (not shown) under control of an electronic system (not shown).

FIG. 2 shows a blood pressure monitoring device as described in the Thede patent. The device includes a wrist assembly placement guide 20, which is generally “U” shaped and includes a hook 21, a pad 22 and an opening 23. The opening 23 is generally circular, and a notch 26 extends from the opening 23 near the hook 21. A pair of guide ribs 24 and 25 encircle the opening 23 and notch 26, and meet at the base of the hook 21. When the placement guide 20 is placed on the patient, the pad 22 contacts the palm side of the wrist of the patient, while hook 21 wraps around the backside of the wrist.

The sensor-based type of device as shown, for example, in FIGS. 1 and 2 is advantageous in many respects, being both accurate with a typical mean correlation of about 0.9 with an indwelling arterial catheter, as well as fast with the ability to calculate systolic pressure, diastolic pressure, and heart rate in about fifteen seconds. Moreover, some versions of the device are able to store and display full pulse waveforms. The sensor-based type of device is also convenient for the patient. Because the device uses a padded sensor placed over the radial artery at the wrist, the patient need not remove any clothing or roll her sleeve to the upper arm. Unlike other techniques such as the cuff, operation with the sensor-type device is smooth with little noise, so it generally does not disturb patients who are resting.

In order to ensure an accurate reading, the sensor should be placed accurately at the distal edge of the radius bone. To assist the patient or a practitioner in attaching the sensor, a placement guide may be provided. As described in the Thede patent, the placement guide is roughly “U”-shaped and approximately cylindrically curved, where the cylindrical axis is perpendicular to the central axis in the “U”. When attached, the “U” fits around a portion of the patient's wrist, with the distal edge of the radius bone located between the prongs of the “U”. In some embodiments, the prongs of the “U” reattach at their distal ends.

The placement guide is provided in different sizes, corresponding to the circumference of the patient's wrist. For instance, an “adult normal” size may correspond to wrist circumferences of about 15 to 18 cm. A “large adult” may correspond to wrist circumferences of about 18 to 22 cm. A “pediatric” may correspond to wrist circumferences of about 11 to 15 cm. These three sizes cover most or all of the normal ranges of wrist sizes of patients.

Because these placement guides come in various sizes in order to accommodate the various ranges in the sizes of patients' arms, three different parts are manufactured, stocked in inventory, and provided with the monitoring device.

### BRIEF SUMMARY OF THE INVENTION

To overcome the disadvantages of the prior art, a placement guide that can accommodate an extended range of wrist circumferences is provided for a sensor-based monitoring device that uses a sensor placed over the radial artery to measure blood pressure. Advantageously, a single placement guide may be used over a large range of patient sizes.

These and other advantages are realized in varying degrees by the various embodiments of the present invention. One embodiment of the present invention is an apparatus for portably and non-invasively monitoring blood pressure of a patient, comprising a body having a first attachment site spaced apart from a second attachment site over an intervening region of the body; a sensor; and a positioning guide. The sensor comprises a support member coupled to the body in proximity to the intervening region; a pressure transducer; and a pressure pulse transmission medium having a sensing surface for contacting tissue of the patient, the pressure transmission medium being supported by the support member and coupled to the pressure transducer for conveying pressure pulses thereto from the sensing surface. The positioning guide is coupled to the body away from the sensor and extending longitudinally toward and beyond the sensor with an arc-like shape generally conformable with a cross-section of an anatomical structure from which blood pressure pulses are detectable, and has an opening for permitting the sensor to pass therethrough. The positioning guide comprises a plurality of longitudinally arranged segments, wherein each longitudinally adjacent pair of the segments are connected by a transverse articulation region having a predetermined shape and a tendency to return to the predetermined shape after flexure; and the segments are less flexible than the articulation region.

Another embodiment of the present invention is a positioning guide for use with a noninvasive device having an operationally projecting sensor for monitoring blood pressure of a patient, comprising a plurality of longitudinally arranged segments, each longitudinally adjacent pair of the segments being connected by a transverse articulation region having a predetermined shape and a tendency to return to the predetermined shape after flexure; and an attachment site-disposed at one of the segments for attaching the positioning guide to the device. The segments are less flexible than the articulation region. At least one of the segments has an opening spaced away from the attachment site for accommodating the projecting sensor. The connected segments extend from the attachment site longitudinally toward and beyond the opening with an arc-like shape generally conformable with a cross-section of an anatomical structure from which blood pressure pulses are detectable.

Another embodiment of the present invention is a positioning guide for use with a noninvasive device having an operationally projecting sensor for monitoring blood pressure of a patient, comprising a curved flexible strip having a predetermined shape and a tendency to return to the predetermined shape after flexure; a plurality of segments of material disposed on and longitudinally spaced apart along the curved flexible strip, each longitudinally adjacent pair of the segments being connected by a transverse section of the curved flexible strip; and an attachment site for attaching the positioning guide to the device. At least one of the segments has an opening spaced away from the attachment site for accommodating the projecting sensor. The connected segments extend from the attachment site longitudinally toward and beyond the opening with an arc-like shape generally conformable with a cross-section of an anatomical structure from which blood pressure pulses are detectable.

Another embodiment of the present invention is a positioning guide for use with a noninvasive device having an operationally projecting sensor for monitoring blood pressure of a patient, comprising a curved flexible polyurethane strip having embedded polyester threads; first, second and third thermoplastic sheets disposed on and longitudinally spaced apart along the polyurethane strip, the segments being less flexible than the polyurethane strip, and each longitudinally adjacent pair of the segments being connected by a transverse section of the polyurethane strip; and an attachment site disposed in proximity to a first end of the first thermoplastic sheet for attaching the positioning guide to the device, the polyurethane strip being absent from about the attachment site. The first thermoplastic sheet further has an aperture in proximity to a second end of the first thermoplastic sheet opposite the first end for accommodating the projecting sensor, the first thermoplastic sheet further having two longitudinal rib sections and the aperture being disposed therebetween and spaced away from the attachment site. The first, second and third sheets extend from the attachment site longitudinally toward and beyond the opening with an arc-like shape generally conformable with a cross-section of an anatomical structure from which blood pressure pulses are detectable.

### BRIEF DESCRIPTION OF THE SEVERAL VIEWS OF THE DRAWINGS

FIG. 1 is a plan drawing of one type of prior art noninvasive blood pressure monitor.

FIG. 2 is a plan drawing of another type of prior art noninvasive blood pressure monitor which includes a placement guide.

FIG. 3 is a perspective edge view of a portable sensor-based device for the noninvasive monitoring of blood pressure, as mounted on the wrist of a patient and which includes an articulated placement guide.

FIG. 4 is a side view of the monitoring device of FIG. 3.

FIG. 5 is a perspective drawing of an articulated placement guide suitable for use with wrist-mounted sensor-based devices for monitoring blood pressure.

FIG. 6 is a side view drawing of the articulated placement guide of FIG. 5.

FIG. 7 is a top view drawing of the articulated placement guide of FIG. 5.

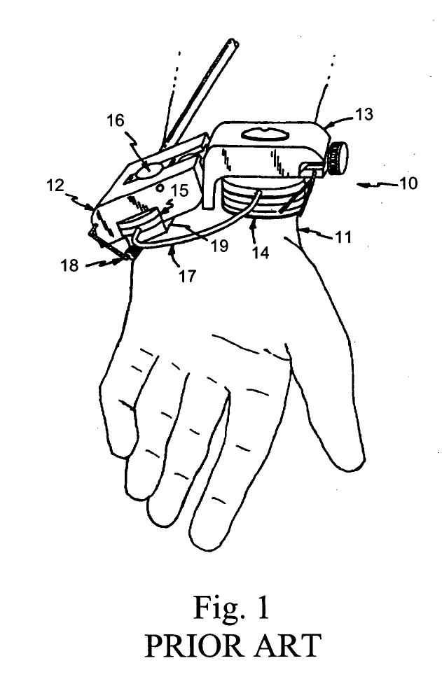

### DETAILED DESCRIPTION OF THE INVENTION

Blood pressure may be determined from a sensor-based monitoring device that non-invasively senses at the surface of a patient's body pressure pulses that are influenced by blood flow in an underlying artery. A user positions the sensor on the wrist over the edge of the radius bone using an articulated placement guide. As varying hold-down pressure is applied so that the properly positioned sensor compresses tissue overlying the artery, pressure pulses are sensed by the sensor to produce data for various purposes, such as calculation of blood pressure and display of pulse waveform. Noninvasive sensor-based monitors for monitoring blood pressure, including systolic pressure, diastolic pressure, and pulse, are described in U.S. Pat. No. 5,797,850 issued Aug. 25, 1998 to Archibald et al., U.S. Pat. No. 5,640,964 issued Jun. 24, 1997 to Archibald et al., and U.S. Pat. No. 6,558,335 issued May 6, 2003, to Thede, which hereby are incorporated herein in their entirety by reference thereto. Placement guides may advantageously be used with these as well as other types of sensor-based monitors.

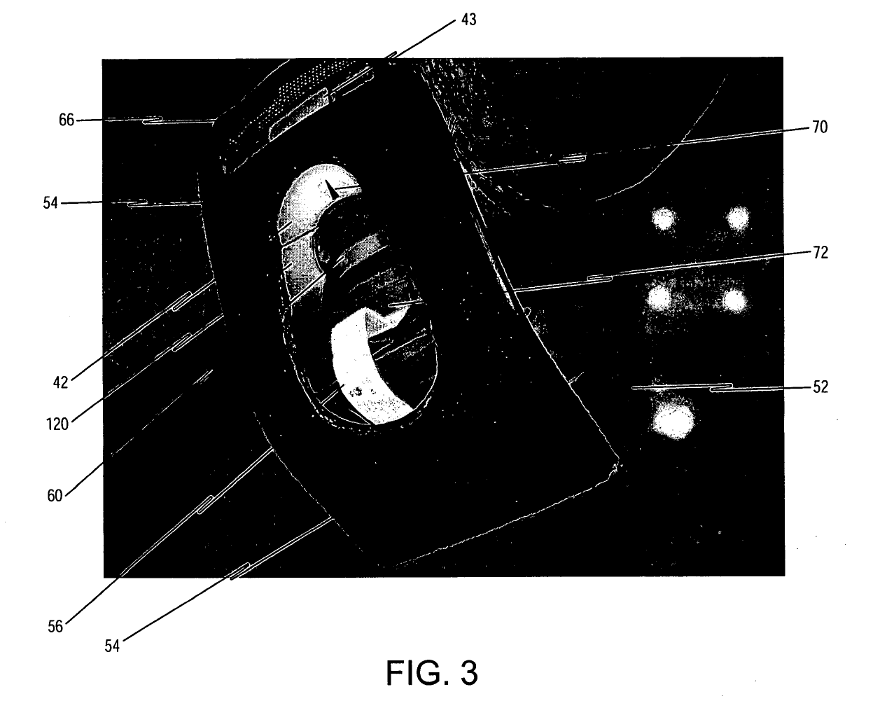

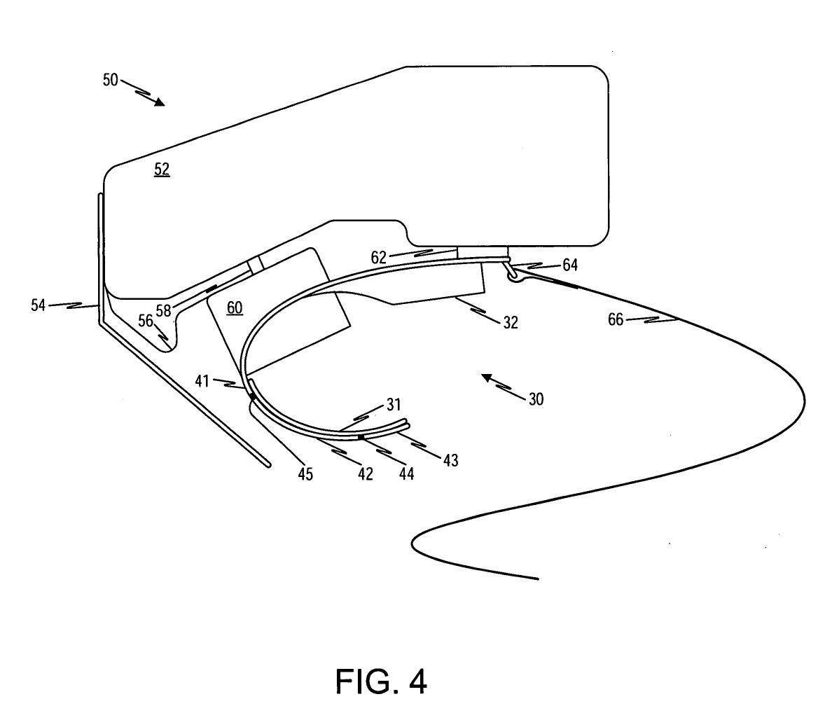

FIG. 3 is a perspective edge view of a portable sensor-based device for the noninvasive monitoring of blood pressure, as mounted on the wrist of a patient and which includes an articulated placement guide. FIG. 4 is a side view of the monitoring device of FIG. 3. The monitoring device 50 has a housing 52, a hold-down pressure generating unit (not shown) contained within the housing 52, and a sensor 60 pivotally coupled to the hold-down pressure generating unit by a sensor rod 58. The hold-down pressure generating unit includes a control circuit, a pneumatic system, and a power source. A user interface panel (not shown) is electrically coupled to the control circuit, and includes controls and numeric indicators. A start/stop switch on the user interface panel is pressed to initiate a monitoring cycle in which a varying force is applied to the radial artery by the hold-down pressure generating unit, and the counter pressure in the radial artery produces a signal that is digitized and used to calculate blood pressure. An illustrative monitoring device is described in further detail in a copending U.S. patent application filed on even date herewith in the name of inventor Kevin R. Evans and entitled “Sensor-based apparatus and method for portable noninvasive monitoring of blood pressure” (Attorney Docket No. 01845.0042-US-01) which hereby is incorporated herein in its entirety by reference thereto.

The monitoring device 50 is secured to the patient in any convenient manner, illustratively by strapping it on with a Velcro® brand strap 66. The ends of the strap 66 are looped through respective anchors 54 and 64, which are attached at or near opposite ends of the monitoring device 50. The anchor 64 is illustratively a U-shaped metal bracket rotatably mounted into a block 62 that projects from the casing 52. The anchor 54 is an elongated strip of flexible material that tends to maintain its shape, such as preformed plastic, which is secured to one end of the casing 52 by any suitable technique such as with glue or mechanical fasteners, and which extends away from the casing 52 either to hold the strap 66 away from the patient's arm when the monitoring device 50 is being slipped onto the patients arm, or to facilitate insertion of the strap 66 through a transverse slot at the distal end of the anchor 54.

To locate the proper position for placement of the sensor 60, the user first palpates the arm with a finger to find the distal edge of the radius bone. The sensor 60 is then placed directly over this point, and the strap 66 is secured snugly. An articulated placement guide 30 that includes articulated segments 41, 42 and 43 helps in the proper placement. The placement guide is attached at one end of the 41 to the casing 52 by the mounting block 62. When the monitoring device 50 is applied to the patient, the placement guide 30 straddles the styloid process bone of the patient and generally guides the sensor 60 into position over the underlying artery and the radius bone. Indicator symbols such as notch symbol 70 on the placement guide segment 42 and arrow symbol 72 on the sensor 60 align to the distal edge of the radius bone when the sensor 60 is properly positioned, and proper placement may be verified tactilely by passing a finger through an elongated access slot in the anchor 54 and an access notch in the placement guide segments 41 and 42, and feeling the distal edge of the radius bone. The access notch extends from a generally circular aperture through which the sensor 60 moves. The portions of segment 42 that flank the aperture and access notch may be thought of as guide ribs which meet within segment 42.

The use of the notch indicator symbol 70 and the arrowhead indicator symbol 72 is somewhat arbitrary, and other shapes conveying a sense of direction could be used as well. Good placement of the sensor over the radius bone causes both indicator symbols 70 and 72 to point to the distal edge of the radius bone. Poor placement causes neither of the indicator symbols 70 and 72 to point to the distal edge of the radius bone. In the event that one of the indicator symbols 70 and 72 is obscured during the positioning process, the other indicator symbol is visible to confirm alignment.

With the monitoring device 50 properly positioned, the monitoring device 50 is switched on and a cycle is initiated by pressing the start/stop switch. As the hold-down pressure generating unit operates, it moves the sensor 60 away from the casing 52 by extending the sensor post 58. The sensor 60 gently exerts pressure against the patient's wrist over the radial artery, while cushion 32 on the placement guide segment 41 and layer 31 extending across whole or parts of placement guide segments 41, 42 and 43 and spanning intervening gaps 44 and 45 gently distribute pressure over other areas of the patient's wrist. The cushion 32 also functions as a pivot point about which the hold-down pressure is applied.

Since the sensor 60 is relatively small compared to such devices as cuffs used with the oscillometric and auscultatory methods, the sensor 60 applies a hold down pressure to only a relatively small area above the underlying artery of the patient. Consequently, blood pressure measurements may be taken with less discomfort to the patient. Because the sensor 60 does not require inflation or deflation, faster and more frequent measurements may be taken. Furthermore, the sensor 60 better conforms to the anatomy of the patient so as to be more comfortable to the patient, and the improved accuracy and repeatability of placement and the automatic application of the hold-down pressure avoids ineffective hold-down cycles and achieves consistent and accurate blood pressure measurements.

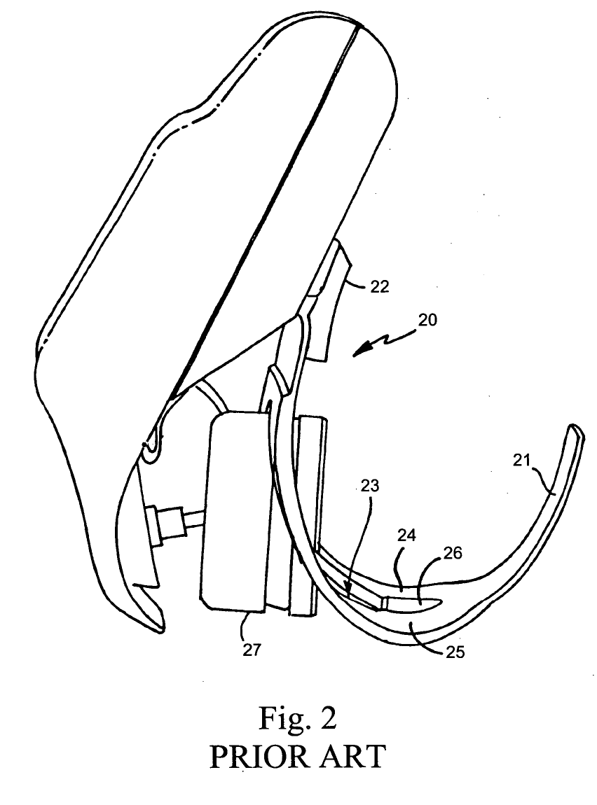

Although placement guides such as the guide 20 (FIG. 2) are typically made of a flexible plastic material having a predetermined shape and a tendency return to that shape when flexed, the range of wrist sizes for which a particular placement guide 20 works is limited. As a result, the placement guide 20 is generally produced in a number of different sizes, which can be interchanged on a monitoring device to accommodate patients of different sizes. However, a disadvantage of using different sized placement guides is the need for keeping all of the different sizes with the measurement device, and the time and inconvenience of removing an inappropriately sized placement guide and installing a correctly sized placement guide.

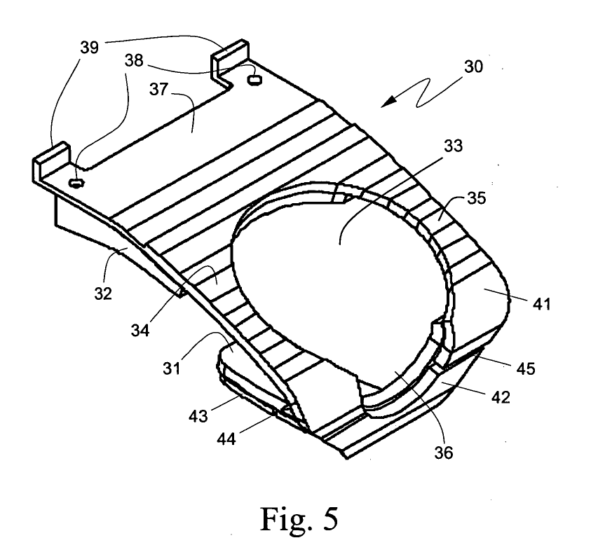

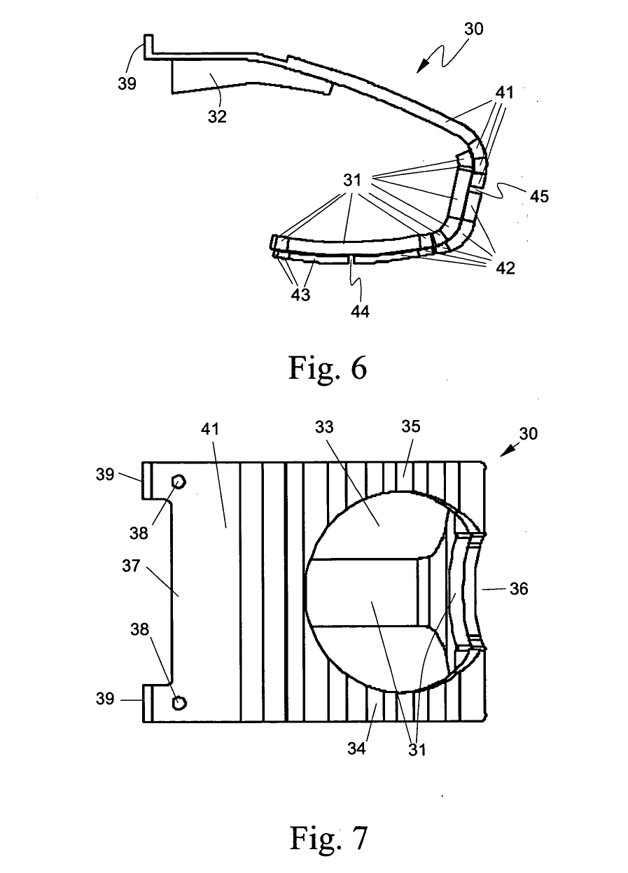

FIGS. 5-7 show three different views of the articulated placement guide 30. The articulated placement guide 30 is divided into three segments 41, 42 and 43, at articulation regions 44 and 45. Because of the articulation regions 44 and 45, the range over which the articulated placement guide 30 can expand to fit the wrist snugly is greater than earlier placement guides, thereby eliminating the need for several different-sized placement guides to cover a full range of the most common patient sizes. In this manner, a single articulated placement guide 30 can accommodate a variety of different patients, resulting in greater convenience and less expense for the monitoring device.

Although three segments are shown in the articulated placement guide 30, it will be appreciated that any suitable number of segments may be used, including two, four, and greater than four.

While the articulation regions 44 and 45 may be formed in any of a variety of different ways, one simple and effective technique is to use a continuous flexible inner layer 31 that extends across all or parts of the three segments 41, 42 and 43. Preferably, the inner layer 31 is a single piece of shaped material that tends to return to its original shape after being flexed, which increases comfort for the patient and strength of the articulated placement guide 30. Alternatively, separate pieces in a discontinuous layout may be used to join adjacent segments. A preferable material for the inner layer 31 is polyurethane with polyester threads embedded in it, which enhances strength, flexibility and durability.

While the articulated placement guide 30 may be made in any of a variety of different ways, one technique is to form the inner layer 31 as a curved polyurethane strip with embedded polyester threads, and then place the strip in a mold for overmolding the inner layer 31 with segments of any suitable plastic such as Kydex® thermoplastic, which is a thermoplastic alloy. Although the plastic segments made with Kydex thermoplastic have some flex, they are generally rigid and have much less flexibility than the polyurethane strip, which makes the articulated placement guide 30 easier to work with during application of the monitoring 50 device to the patient. However, if desired an even more rigid material may be used for the segments.

An articulated placement guide such as the guide 30 having 3 segments and two articulation regions may be dimensioned as follows to fit a range of wrist circumferences from about 11 cm to about 22 cm: length of about 13 cm, minimum width of about 1.3 cm, maximum width of about 4.8 cm, minimum segment thickness about 1.27 mm (50 mils), maximum segment thickness (generally about the aperture 33) of about 2.29 mm (90 mils), and thickness of the inner layer 31 of about 2.54 mm (100 mils). The range of wrist sizes from about 11 cm to about 22 cm is sufficient to include a broad spectrum of patients, ranging from children weighing as little as 43 pounds to obese and bariatric patients. This range covers about 95% of all patients.

The cushion 32 is affixed to the inside of segment 41 to provide comfort for the patient and to stabilize the monitoring device on the patient's wrist. The cushion 32 may be made from any suitable material.

The articulated placement guide 30 further has a sensor aperture 33 which allows the sensor 60 (FIG. 3) to pass through the articulated placement guide 30 and contact the patient. Guide ribs 34 and 35 are provided on the sides of the sensor aperture 33. A notch 36 extends from the aperture 33 so that during placement of the monitoring device on the wrist, the patient or practitioner adjusts the device until he or she can feel the distal end of the radius bone with a finger placed through the notch 36 to ensure proper placement. Segment 41 has a ledge 37 for engagement with other mechanical parts of the device, as well as screw holes 38 and ridges 39, for attachment to the monitoring device. It will be appreciated that other methods for attachment may be used in addition to, or instead of elements 37, 38 and 39. The attachment may be removable or permanent.

The articulated placement guide 30 may be made from a variety of suitable materials, including plastics, rubbers, and metals. In one variation, a thin springy stainless steel may be used for the inner layer 31, with a suitable segmented overcoat of a protective and cushioning material. In other variations, the articulated placement guide hinges may be realized by a continuous sheet of flexible material that has a predetermined shape and a tendency to return to that shape when flexed, but which is thinned or otherwise made more flexible in limited predetermined places to form the articulation regions.

It will be appreciated that the articulated placement guide 30 may be used in monitoring devices that automatically adjust the hold-down force between the sensor and the patient's wrist, as well as measurement in which the hold-down force is adjusted manually. The sensor in such devices may be unitary, or various components of the sensor may be distributed elsewhere in the device. Where the sensor includes a pressure transducer, for example, the pressure transducer may be mounted to a supporting member of the sensor that also supports the pressure transmission medium containing the sensing surface, or may be mounted to a supporting member elsewhere in the device and placed in fluid communication with the sensing surface through a fluid-filled tube.

It will be appreciated that although the articulated placement guide is described herein in the context of a wrist-mounted monitoring device, the monitoring device and the associated articulated placement guide may be designed for use with other anatomical structures on which noninvasive monitoring for blood pressure may be performed, including the inside elbow, the ankle, and the top of the foot.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. An apparatus for portably and non-invasively monitoring blood pressure of a patient, comprising:
a body having a first attachment site spaced apart from a second attachment site over an intervening region of the body;
a sensor comprising:
a support member coupled to the body in proximity to the intervening region;
a pressure transducer; and
a pressure pulse transmission medium having a sensing surface for contacting tissue of the patient, the pressure transmission medium being supported by the support member and coupled to the pressure transducer for conveying pressure pulses thereto from the sensing surface; and
a positioning guide coupled to the body away from the sensor and extending longitudinally toward and beyond the sensor with an arc-like shape generally conformable with a cross-section of an anatomical structure from which blood pressure pulses are detectable, the positioning guide having an opening for permitting the sensor to pass therethrough;
the positioning guide comprising a plurality of longitudinally arranged segments, wherein:
each longitudinally adjacent pair of the segments are connected by a transverse articulation region having a predetermined shape and a tendency to return to the predetermined shape after flexure; and
the segments are less flexible than the articulation region.

2. The apparatus of claim 1 wherein the positioning guide comprises:
a curved polyurethane strip having embedded polyester threads;
wherein each of the segments comprises a sheet of plastic material; and
wherein the plastic sheets are disposed on the curved polyurethane strip and spaced apart from one another so that the articulation region between each longitudinally adjacent pair of the segments is formed by a transverse section of the curved polyurethane strip.

3. The apparatus of claim 2 wherein the plurality of segments in the positioning guide is limited to two segments.

4. The apparatus of claim 2 wherein the plurality of segments in the positioning guide is limited to three segments.

5. The apparatus of claim 4 wherein the anatomical structure is a wrist, further comprising:
a wrist engaging member having a first end coupled to the first attachment site, and a second end coupled to the second attachment site;
wherein the segments in the positioning guide are configured to accommodate a range of wrist sizes from about 11 cm to about 22 cm.

6. A positioning guide for use with a noninvasive device having an operationally projecting sensor for monitoring blood pressure of a patient, comprising:
a plurality of longitudinally arranged segments, each longitudinally adjacent pair of the segments being connected by a transverse articulation region having a predetermined shape and a tendency to return to the predetermined shape after flexure; and
an attachment site disposed at one of the segments for attaching the positioning guide to the device;
wherein the segments are less flexible than the articulation region;
wherein at least one of the segments has an opening spaced away from the attachment site for accommodating the projecting sensor; and
wherein the connected segments extend from the attachment site longitudinally toward and beyond the opening with an arc-like shape generally conformable with a cross-section of an anatomical structure from which blood pressure pulses are detectable.

7. The positioning guide of claim 6 wherein the plurality of segments is limited to two segments.

8. The positioning guide of claim 6 wherein the plurality of segments is limited to three segments.

9. The positioning guide of claim 6 further comprising:
a curved strip of flexible material having a predetermined shape and a tendency to return to the predetermined shape after flexure;
wherein each of the segments comprises a sheet of material less flexible than the curved strip; and
wherein the sheets are disposed on the curved strip and spaced apart from one another so that the articulation region between each longitudinally adjacent pair of the segments is formed by a transverse section of the curved strip.

10. The positioning guide of claim 6 further comprising:
a curved polyurethane strip having embedded polyester threads;
wherein each of the segments comprises a sheet of plastic material; and
wherein the plastic sheets are disposed on the curved polyurethane strip and spaced apart from one another so that the articulation region between each longitudinally adjacent pair of the segments is formed by a transverse section of the curved polyurethane strip.

11. The positioning guide of claim 6 further comprising:
a curved stainless steel strip;
wherein each of the segments comprises a sheet of material; and
wherein the sheets are disposed on the curved stainless steel strip and spaced apart from one another so that the articulation region between each longitudinally adjacent pair of the segments is formed by a transverse section of the curved stainless steel strip.

12. The positioning guide of claim 6 wherein:
each of the segments is a part of an elongated strip of flexible material; and
the articulation region between each longitudinally adjacent pair of the segments is a relatively thin transverse section of the elongated strip.

13. The positioning guide of claim 6 wherein:
the articulation region between each longitudinally adjacent pair of the segments comprises flexible material having a predetermined shape and a tendency to return to the predetermined shape after flexure; and
each of the segments comprises a sheet of material less flexible than the flexible material between each longitudinally adjacent pair of the segments.

14. A positioning guide for use with a noninvasive device having an operationally projecting sensor for monitoring blood pressure of a patient, comprising:
a curved flexible strip having a predetermined shape and a tendency to return to the predetermined shape after flexure;
a plurality of segments of material disposed on and longitudinally spaced apart along the curved flexible strip, each longitudinally adjacent pair of the segments being connected by a transverse section of the curved flexible strip; and
an attachment site for attaching the positioning guide to the device;
wherein at least one of the segments has an opening spaced away from the attachment site for accommodating the projecting sensor; and
wherein the connected segments extend from the attachment site longitudinally toward and beyond the opening with an arc-like shape generally conformable with a cross-section of an anatomical structure from which blood pressure pulses are detectable.

15. The positioning guide of claim 14 wherein:
each of the segments comprises a sheet of material less flexible than the curved flexible strip; and
wherein the sheets are disposed on the curved flexible strip and spaced apart from one another.

16. The positioning guide of claim 14 wherein:
the curved flexible strip is a curved polyurethane strip having embedded polyester threads;
each of the segments comprises a sheet of plastic material; and
the plastic sheets are disposed on the curved polyurethane strip and spaced apart from one another.

17. The positioning guide of claim 14 wherein:
the curved flexible strip comprises a curved stainless steel strip;
wherein each of the segments comprises a sheet of material; and
wherein the sheets are disposed on the curved stainless steel strip and spaced apart from one another.

18. A positioning guide for use with a noninvasive device having an operationally projecting sensor for monitoring blood pressure of a patient, comprising:
a curved flexible polyurethane strip having embedded polyester threads;
first, second and third thermoplastic sheets disposed on and longitudinally spaced apart along the polyurethane strip, the segments being less flexible than the polyurethane strip, and each longitudinally adjacent pair of the segments being connected by a transverse section of the polyurethane strip; and
an attachment site disposed in proximity to a first end of the first thermoplastic sheet for attaching the positioning guide to the device, the polyurethane strip being absent from about the attachment site;
wherein the first thermoplastic sheet further has an aperture in proximity to a second end of the first thermoplastic sheet opposite the first end for accommodating the projecting sensor, the first thermoplastic sheet further having two longitudinal rib sections and the aperture being disposed therebetween and spaced away from the attachment site; and
wherein the first, second and third sheets extend from the attachment site longitudinally toward and beyond the opening with an arc-like shape generally conformable with a cross-section of an anatomical structure from which blood pressure pulses are detectable.

19. The positioning guide of claim 18 wherein the attachment site comprises a plurality of holes for attachment of the positioning guide to the device with screws.

20. The positioning guide of claim 19 further comprising a cushion disposed about the attachment site.
